(19) United States
(12) Patent Application Publication
(10) Pub. No.: US 2011/0251450 A1
(43) Pub. Date: Oct. 13, 2011

(54) Method and Device for Attachment of an Inflow Conduit to the Heart and to a Pump

(76) Inventors: Francis D. Pagani, South Lyon, MI (US); Takeshi Tsubouchi, Dexter, MI (US)

(21) Appl. No.: 12/758,100

(22) Filed: Apr. 12, 2010

(51) Int. Cl. A61M 1/12

(52) U.S. Cl. 600/16

(57) ABSTRACT

Apparatus connects a pump to a chamber of a heart. A cuff attaches to the heart, the cuff having a flange portion for attaching to a surface of the heart and a sleeve portion extending from the flange portion. An inflow conduit has a first end for inserting into the heart, an intermediate portion for attaching to the sleeve portion of the cuff, and a second end for conveying blood from the heart to the pump. An expansion tool is releasably mounted with the inflow conduit to slide longitudinally within the inflow conduit. The expansion tool has a blunt tip for punching through the heart into the chamber at a distal end and a manual grip at a proximal end for pulling the expansion tool out from the inflow conduit. The expansion tool has a sliding seal substantially adjacent the blunt tip so that after punching into the heart and joining the inflow conduit to the sleeve portion of the cuff, the expansion tool can be withdrawn from the inflow conduit resulting in gradual entry of blood into the first end and withdrawal of air from the second end.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates in general to cardiac assist systems, and, more specifically, to methods and a tool for inserting and attaching a blood flow conduit to a patient's heart.

A heart pump system known as a left ventricular assist system (LVAS) can provide long term patient support with an implantable pump associated with an externally-worn pump control unit and batteries. The LVAS improves circulation throughout the body by assisting the left side of the heart in pumping blood. One such system is the DuraHeart® LVAS system made by Terumo Heart, Inc., of Ann Arbor, Mich. The DuraHeart® system employs a centrifugal pump with a magnetically levitated impeller to pump blood from the left ventricle to the aorta. An inflow conduit comprised of a small titanium tube connects the left ventricle to the pump. The inflow conduit connects to the heart via an attachment cuff and to the pump via a threaded fitting. An example of such an inflow conduit is shown in U.S. Pat. No. 7,048,681, incorporated by reference herein in its entirety.

When they were first introduced, ventricular assist devices were implanted during a surgery in which a blood perfusion system was used so that the beating of the heart could be stopped during attachment of the inflow conduit to the ventricle in order to avoid blood loss and air introduction. This is commonly referred to as an “on-pump” procedure. However, certain risks are associated with an on-pump procedure, so off-pump procedures have been investigated. U.S. Pat. No. 6,726,648, for example, uses a check valve in an inflow cuff to reduced blood loss prior to insertion of the inflow conduit. However, the '648 patent has a disadvantage that air is introduced into the pump circuit during installation of the inflow conduit, and extra steps must be taken to remove the air so that it cannot enter the bloodstream of the patient.

Thus, it would be desirable to provide improved methods and tools for attaching an inflow conduit in an off-pump procedure that avoids the introduction of air into the pump circuit.

### SUMMARY OF THE INVENTION

The present invention achieves the installation of an inflow conduit onto a beating heart while limiting blood loss and preventing the introduction of air into the pump circuit.

In one aspect of the invention, an apparatus is provided for connecting a pump to a chamber of a heart. A cuff attaches to the heart, the cuff having a flange portion for attaching to a surface of the heart and a sleeve portion extending from the flange portion. An inflow conduit has a first end for inserting into the heart, an intermediate portion for attaching to the sleeve portion of the cuff, and a second end for conveying blood from the heart to the pump. An expansion tool is releasably mounted with the inflow conduit to slide longitudinally within the inflow conduit. The expansion tool has a blunt tip for punching through the heart wall into the chamber at a distal end and a manual grip at a proximal end for pulling the expansion tool out from the inflow conduit. The expansion tool has a sliding seal substantially adjacent the blunt tip so that after punching into the heart and joining the inflow conduit to the sleeve portion of the cuff, the expansion tool can be withdrawn from the inflow conduit resulting in gradual entry of blood into the first end and withdrawal of air from the second end.

### BRIEF DESCRIPTION OF THE DRAWINGS

FIG. 1 is a front view of a left ventricular assist system implanted into a patient.

FIG. 2 is a partial cross section showing an attachment procedure according to the present invention.

FIG. 3 is a side view of an inflow conduit being installed through a cuff.

FIG. 4 is a side view of a first embodiment of an expansion tool of the invention.

FIG. 5 is a top view of the expansion tool of FIG. 4.

FIG. 6 is a side view showing the expansion tool installed into the inflow conduit.

FIGS. 7 to 11 illustrate various steps in a surgical procedure to implant the ventricle assist device.

FIG. 12 is a side view showing an alternate shape for the blunt tip of the expansion tool.

FIG. 13 is a side, cross-sectional view of an embodiment of the expansion tool and inflow conduit using a tapered screw mechanism to expand the tip.

FIG. 14 is a side, cross-sectional view of an embodiment of the expansion tool and inflow conduit using a wedge mechanism, with the tip in a contracted configuration.

FIG. 15 is a side, cross-sectional view of the expansion tool and inflow conduit of FIG. 14, with the tip in an expanded configuration.

FIGS. 16 to 19 are cross-sectional views showing surgical use of the tool of the present invention.

FIG. 20 is a side view showing an alternative embodiment wherein the blunt tip does not expand or contract.

FIGS. 21 and 22 illustrate an alternative embodiment using a tapered screw mechanism to expand the tip.

FIG. 23 is a cross-sectional view of a flap valve installed on the end of the inflow conduit, with the flap valve open.

FIG. 24 is a cross-sectional view of the flap valve of FIG. 23 in the closed position.

FIG. 25 is a perspective view of the flap valve of FIG. 23 in the open position.

FIG. 26 is a perspective view of the flap valve of FIG. 23 in the closed position.

### DETAILED DESCRIPTION OF PREFERRED EMBODIMENTS

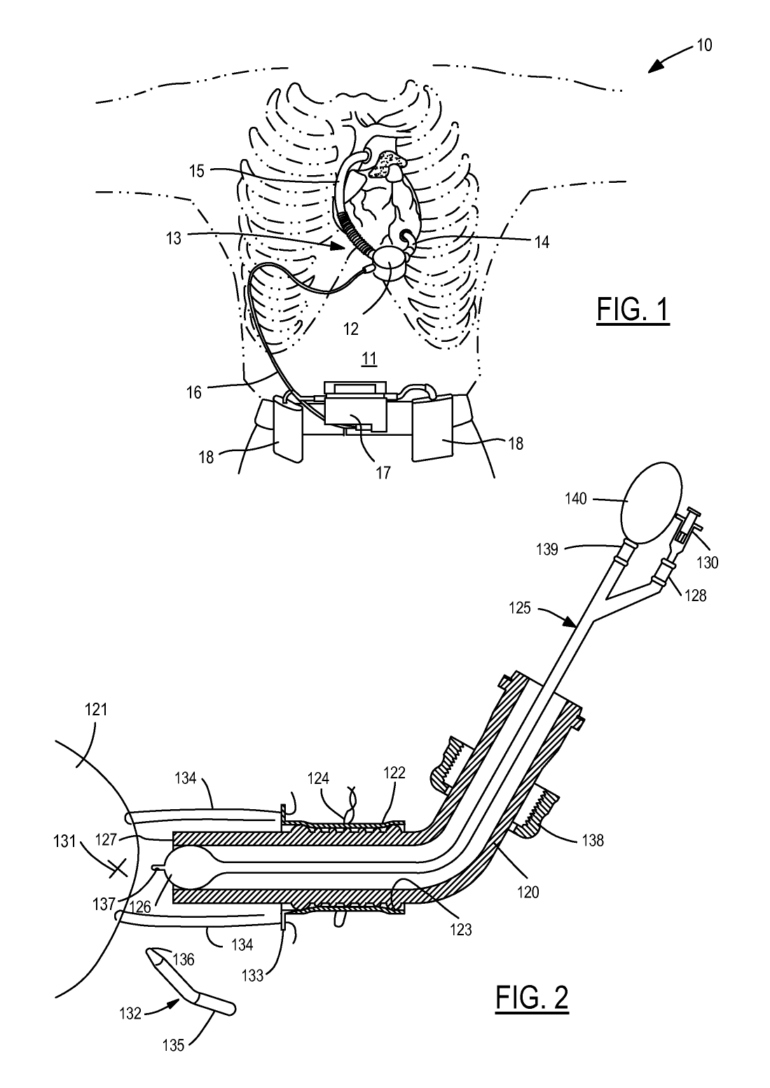

Referring to FIG. 1, a patient 10 is shown in fragmentary front elevational view. Surgically implanted into the patient's abdominal cavity 11 is the pumping portion 12 of a ventricular assist device, generally referenced with the numeral 13. The ventricular assist device 13 includes an inflow conduit 14 conveying blood from the patient's left ventricle into the pumping portion 12, and an outflow conduit 15 conveying blood from the pumping portion 12 to the patient's ascending thoracic aorta. From the pumping portion 12, a power cable 16 extends outwardly of the patient's body via an incision to a compact controller 17. A power source, such as a battery pack worn on a belt about the patient's waist, and generally referenced with the numeral 18, is connected with controller 17.

Each of the conduits 14 and 15 may include a tubular metallic housing proximate the pumping portion 12 which may connect to elongated segments extending to the heart and ascending aorta, respectively. At the end of inflow conduit 14 connected to the patient's heart (preferably at the apex of the left ventricle), and at the end of outflow conduit 15 connected to the ascending thoracic aorta, the conduits are generally attached to the natural tissue by sutures through the use of a sewing ring or cuff so that blood flow communication is established and maintained. The distal end of the inflow conduit 14 is inserted through the ventricle wall and into the heart in order to establish blood flow from the heart to the pumping portion 12. As in the DuraHeart® LVAS system, pumping portion 12 may be comprised of a centrifugal pump with a magnetically levitated impeller.

One embodiment of a first inventive procedure of the present invention is illustrated in FIG. 2 wherein an inflow conduit 120 is to be attached to a heart 121 in an off-pump procedure. Initially, an apical cuff 122 is placed over conduit 120 at an optimum position over retention features 123, and is loosely retained by tying a loop of thread 124 around cuff 122. The cuff includes a tubular portion for receiving inflow conduit 120 and a flange portion for attaching to the heart. The loose loop keeps cuff 122 in position during the insertion of conduit 120 but allows repositioning of cuff 122 is to accommodate adjustments in the final positioning of cuff 122 to attach it to heart 121.

An inflating catheter (such as a Foley catheter) 125 is inserted into conduit 120 and inflated such that an inflation member 126 is sealed against a distal end 127 of conduit 120. Catheter 125 has an internal inflation lumen (not shown) in communication with a coupling or valve 128 that receives a syringe to manually supply or remove an inflation fluid as known in the art. By positioning inflation member 126 at distal end 127 and then inflating, it presses against and blocks distal end 127 to seal it, thereby ensuring that a minimum amount of air will be trapped during insertion into the ventricle of heart 121.

A cut 131 (in the shape of a cross or other desired configuration) is made to partially incise the heart tissue in a selected position of the apex over the ventricle. Cut 131 passes through a majority of the tissue depth but does not penetrate fully into the ventricle, so there is no loss of blood through cut 131. It prevents tearing and enables the final penetration of the hole to be made using a blunt tool 132 (such as a dilator).

After cut 131 is made, the flange 133 of cuff 122 is loosely coupled by a plurality of sutures 134 to the heart wall at positions around cut 131. The loose suturing keeps cuff 122 aligned during the attachment procedure. Dilator 132 has a proximal handle 135 and a blunt distal tip 136. With conduit 120 and cuff 122 in a ready position, the surgeon manipulates dilator 132 to pierce a hole through the remaining heart wall. Dilator 132 is pulled out and then conduit 120 containing catheter 125 is inserted through the hole in the heart wall and into the ventricle. The hole in the heart wall can be temporarily plugged by a finger or thumb of the surgeon to minimize blood loss after dilator 132 is removed and before conduit 120 is inserted.

With conduit 120 inserted through the heart wall, sutures 134 are tightened to closely attach cuff 122 to heart 121. During the piercing of heart 121 and the insertion of conduit 120, the apex of heart 121 is preferably kept in an elevated position in order to minimize the migration of air into the bloodstream.

Catheter 125 includes a second internal lumen (not shown) communicating between an opening 137 and a coupling or valve 139 that functions as a drain lumen. A bulb or pouch 140 is attached to coupling 139 and provides a receptacle for a mixture of air and blood that is drained from within the ventricle prior to the removal of catheter 125. Thus, suction may be applied via the second lumen after final attachment of cuff 122 so that any air around the distal end of conduit 120 is removed, and then inflation member 126 is deflated by an amount sufficient to allow it to be slidingly removed from conduit 120. The surgeon can manually control the degree to deflation so that inflation member 126 slides against the interior of conduit 120 with a light pressure that maintains a seal without harming any coating applied to conduit 120.

Once catheter 125 is fully extracted from conduit 120, the surgeon may use a finger or thumb to cover the proximal edge of conduit 120 to prevent an outflow of blood. Conduit 120 is then attached to a pump (not shown) using threaded coupling 138. With conduit 120 in its final position, loop 124 and any additional sutures, as desired, are tightened to fully secure cuff 122 to conduit 120. After the pump is de-aired by allowing blood to flow into it, the pump is attached to an outflow conduit that connects to an artery (e.g., the aorta), thereby completing the pumping circuit.

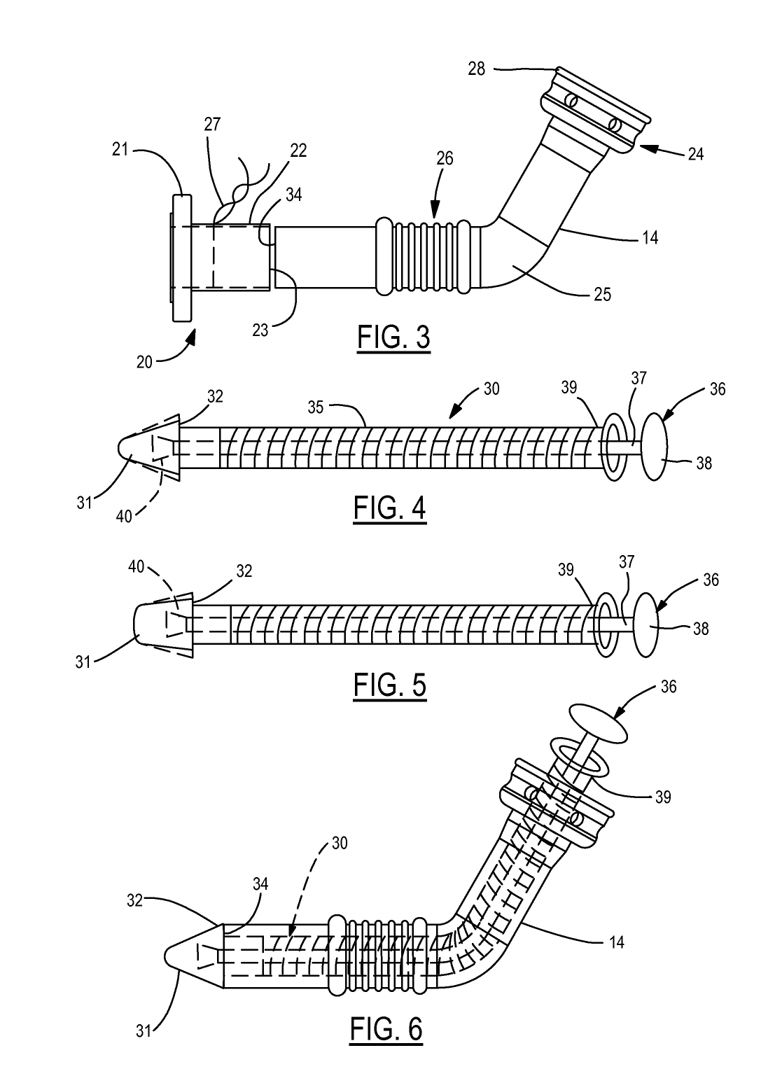

FIG. 3 shows inflow conduit 14 and a cuff 20 in greater detail. Cuff 20 includes a flange portion 21 and a sleeve portion 22, preferably comprised of woven polyester as known in the art. Flange portion 21 is sutured onto an outside surface of the heart (e.g., at the apex in order to provide entry into the left ventricle). Inflow conduit 14 has a distal end 23 for inserting through both the sleeve portion 22 and the flange portion 21 of cuff 20 into the left ventricle. Inflow conduit 14 includes a plurality of protrusions or ribs 26 that slide into sleeve portion 22 when inflow conduit 14 is fully inserted. A plurality of sutures 27 are looped around sleeve portion 22 to compress the cuff around protrusions 26 and provide a seal against leakage of blood. Protrusions 26 provide a locking mechanism for retaining inflow conduit 14 within cuff 20. At a proximal end 24 of inflow conduit 14, a threaded coupling 28 is is provided for attaching to a mating coupler on the pump body.

The present invention uses an expansion or plunger tool for releasably mounting within the inflow conduit. In some preferred embodiments, the distal end of the tool expands and contracts under manual control for selectably retaining in or removing the tool from the inflow conduit. A first embodiment is shown in FIGS. 3 and 4 wherein a blunt tip 31 is located at a distal end of an expansion tool 30 for punching through the heart into the left ventricle or other chamber of the heart. In use, expansion tool 30 is mounted through the inside of inflow conduit 14, as shown in FIG. 5. Blunt tip 31 preferably has an expandable base 32 with an expanded configuration shown by dashed lines in FIGS. 3 and 4 and a contracted configuration shown by solid lines in FIGS. 3 and 4. In the contracted configuration, base 32 has an outside diameter such that a sliding seal is formed between an inside cylindrical surface of inflow conduit 14 and base 32. When in the expanded configuration, base 32 has a diameter greater than or equal to an outside diameter of inflow conduit 14 so that expandable base 32 covers a forward edge 34 of inflow conduit 14. When expansion tool 30 is attached to inflow conduit 14 as shown in FIG. 5, the expanded configuration allows a surgeon to grasp inflow conduit 14 and apply pressure against blunt tip 31 to pierce the heart tissue during emplacement.

In order to follow the curved path within the inside of inflow conduit 14, expansion 30 includes a flexible body 35 extending between blunt tip 31 and a proximal end 36. Flexible body 35 may be comprised of a coiled wire or other types of articulating surfaces such as are well known for producing surgical cannulae. Flexible body 35 has a hollow interior which receives an adjustment member 37 that joins blunt tip 31 to a control handle 38. Flexible body 35 also includes a gripping portion 39 that extends outwardly from inflow conduit 14 so that a surgeon can manually grasp expansion tool 30 in order to pull it out from inflow conduit 14.

By reducing the outside diameter of expandable base 32, expansion tool 30 can be withdrawn from inflow conduit 14 after emplacement in the heart. Withdrawal of tool 30 results in a sliding seal between blunt tip 31 and inflow conduit 14 which achieves gradual entry of blood into the distal end 23 of inflow conduit 14 and gradual withdrawal of air from proximal end 24 as expansion tool 30 is pulled out. In the embodiment shown in FIGS. 3-5, blunt tip 31 includes a wedge mechanism 40 controlled from handle 38 via adjustment member 37 as described in greater detail below. As evidenced by the differences in the profile of blunt tip 31 between the side view of FIG. 3 and top view of FIG. 4, tip 31 may have a duck-bill shape that provides a rounded tip well adapted to piercing the heart tissue without causing significant tissue damage.

A general surgical procedure using the expansion tool of the present invention is shown in FIGS. 6-10. FIG. 6 shows a heart 41 having a chamber such as a left ventricle 42. Cuff 20 is attached by suturing of the flange (i.e., sewing ring) to the apex of heart 41. Once cuff 20 is joined to the heart in this conventional manner, a partial cut is preferably made in the heart tissue at the center of cuff 20 using a knife 43. The procedure of FIGS. 6-10 can also be modified such that the cuff and conduit attachment is done in a manner similar to that disclosed in the embodiment of FIG. 2. In other words, cuff 20 can alternatively be loosely pre-assembled onto conduit 14 and loosely sutured onto the heart before conduit 14 is inserted through the heart wall.

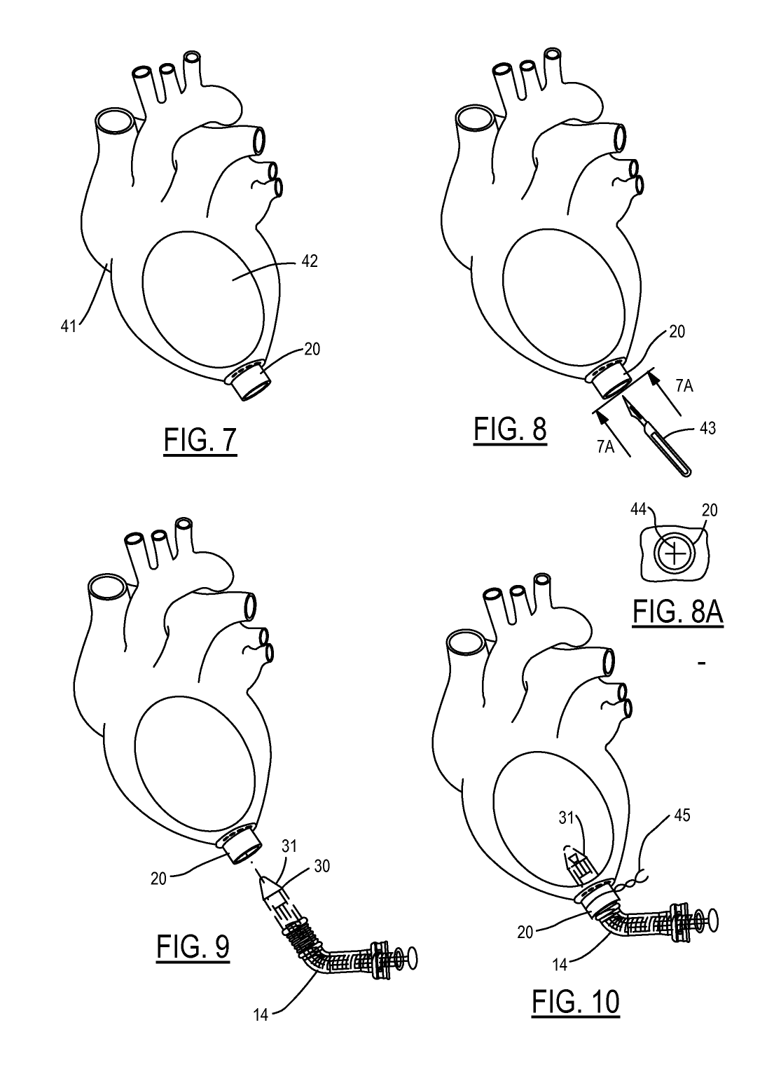

As shown in FIG. 7A, the cut may preferably be in the shape of a cross 44. Cross 44 is cut to a partial depth of the thickness of the heart tissue in order to weaken it for easier punching through by the expansion tool but not yet breaching the heart tissue so that no blood is lost even though the heart continues to beat.

As shown in FIG. 8, inflow conduit 14 together with attached expansion tool 30 are brought into alignment with cuff 20 whereby tip 31 is pressed against partial cut 44, and then force may be applied in order to puncture through the remaining heart tissue. Inflow conduit 14 is advanced until the ribbed portion is inside the sleeve portion of cuff 20 so that sutures 45 can be tied off around the sleeve portion to lock inflow cuff 14 in place and to prevent blood loss between cuff 20 and inflow conduit 14. Since blunt tip 31 is in an expanded configuration, no blood yet enters inflow conduit 14.

The expansion mechanism within blunt tip 31 is adjusted so that blunt tip 31 assumes its contracted configuration, thereby allowing expansion tool 30 to be pulled out as shown in FIG. 10. In the contracted configuration, a sliding seal is provided between blunt tip 31 and the interior of inflow conduit 14. As expansion tool 30 is withdrawn, blood gradually enters inflow conduit 14 at its distal end and air is gradually withdrawn at the proximal end of inflow conduit 14. Once expansion tool 30 is clear from inflow conduit 14, coupler 27 and inflow conduit 14 can be quickly and easily attached to a mating connector on pump 46. If necessary, the surgeon may block the proximal end of inflow conduit 14 with a thumb to prevent any significant blood loss until the connection is finally made. Pump 46 is preferably primed (e.g., with saline) prior to connecting with inflow conduit 14.

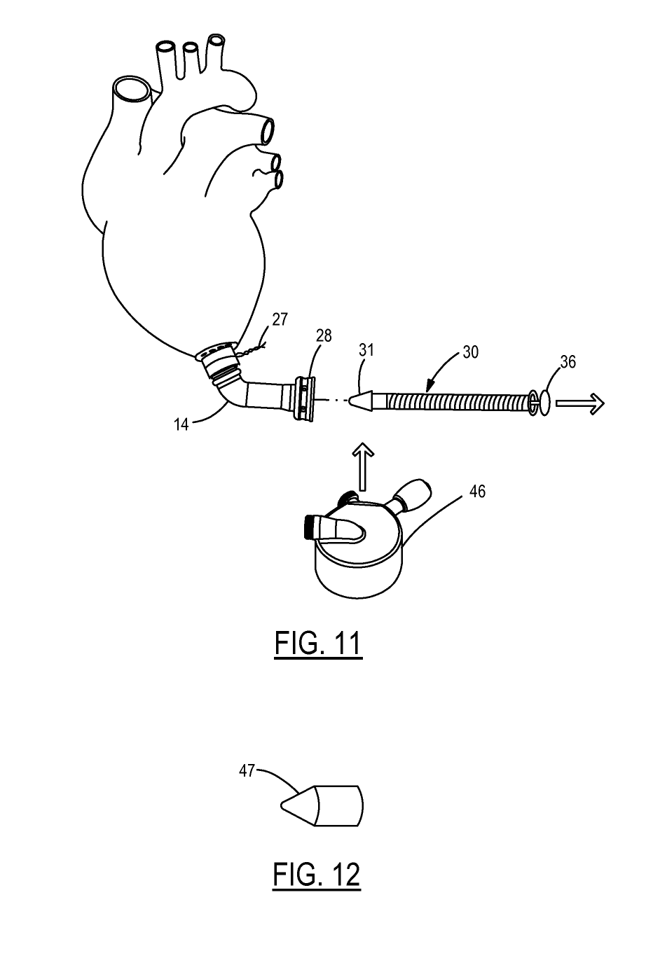

As shown in FIG. 11, the blunt tip may also have a conical shape 47 as an alternative to the duck-bill shape. Other gradually tapered (i.e., generally pointed) shapes are also possible.

FIG. 12 shows a cross-section of an alternative embodiment of the invention wherein a tapered screw mechanism controls expansion and contraction of the tip. An expansion tool 50 is shown installed in inflow conduit 14. A main body 51 includes an expandable tip 52, providing an interior recess 53, a cylindrical section 54, and a gripping collar 55. Main body 51 may, for example, be comprised of a single piece of reinforced silicone rubber or may be made in composite sections using coiled wire as previously discussed. Extending through cylindrical section 54 is an adjustment member 56 having a threaded frustoconical head 57, a shaft 58, and an adjustment knob 59. Head 57 protrudes into recess 53 having matching threads and a diameter that tapers to less than the diameter of head 57. When adjustment member 56 is rotated to advance head 57 into recess 53, tip 52 expands until reaching its expanded configuration which covers the distal end of inflow conduit 14.

Inflow conduit 14 is typically provided with an anti-coagulant coating which may be accidentally scrubbed off if not handled properly. By expanding to a diameter greater than or equal to the diameter of inflow conduit 14, tip 52 protects the anti-coagulant coating during insertion of inflow conduit 14 into the heart so as not to allow contact with other surfaces to scrub off the coating. After insertion and attachment of inflow conduit 14 to the apical cuff, adjustment knob 59 is rotated in a direction to withdraw head 57 from recess 53 so that the diameter of base 60 of tip 52 is reduced to its contracted configuration thereby producing a gap between base 60 and the inside diameter of inflow conduit 14 so that during withdraw of expansion tool 50 a seal is maintained that substantially prevents air from entering the gap and that prevents scrubbing of the anti-coagulant coating from inflow conduit 14.

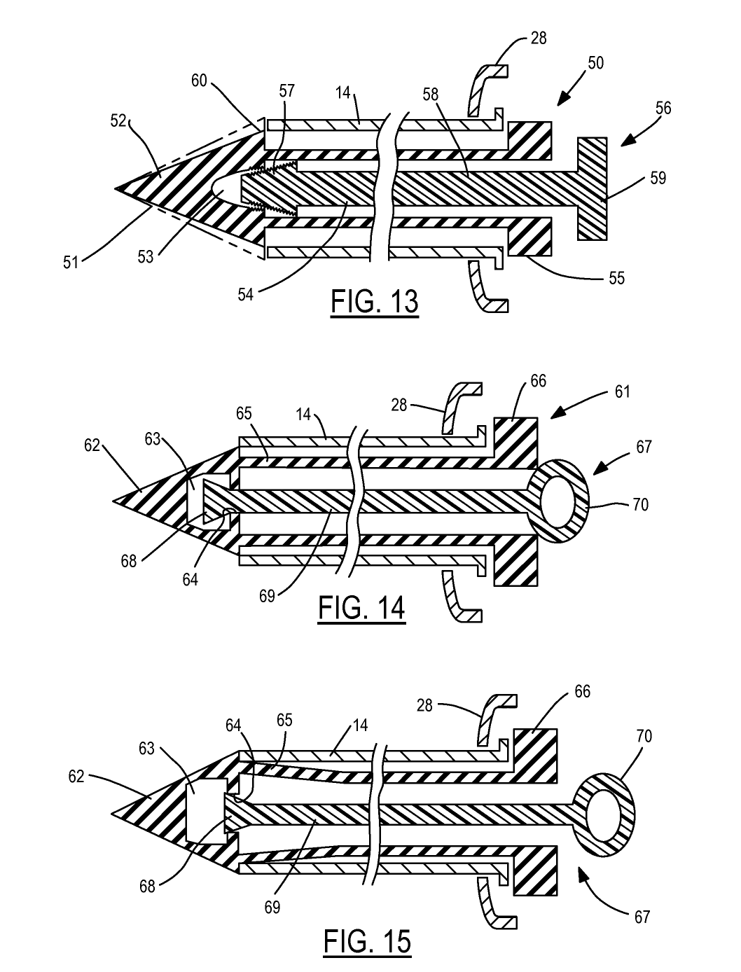

FIGS. 13 and 14 shows an embodiment using the wedge mechanism in greater detail. In this example, expansion tool 61 includes a tip 62 having an internal recess 63 defining an entry bore 64. Cylindrical section 65 joins tip 62 with a grip 66 at opposite ends. An adjustment member 67 has a wedge end 68 joined by a shaft 69 to a push/pull ring 70. FIG. 13 shows expansion tool 61 in its contracted configuration with wedge 68 fully inserted in recess 63. The diameter of shaft 69 matches the diameter of entry bore 64 so that tip 62 is not expanded. As shown in FIG. 14, by pulling adjustment member 67 outward (e.g., by manually pulling on ring 70), wedge 68 is pulled into entry bore 64 resulting in expansion of the base of tip 62 to the expanded configuration wherein tip 62 covers and protects the end of inflow conduit 14. To remove expansion tool 61 from inflow conduit 14, adjustment member 67 is pushed back into the tool so that wedge 68 re-enters recess 63 and the base of tip 62 reassumes its contracted configuration.

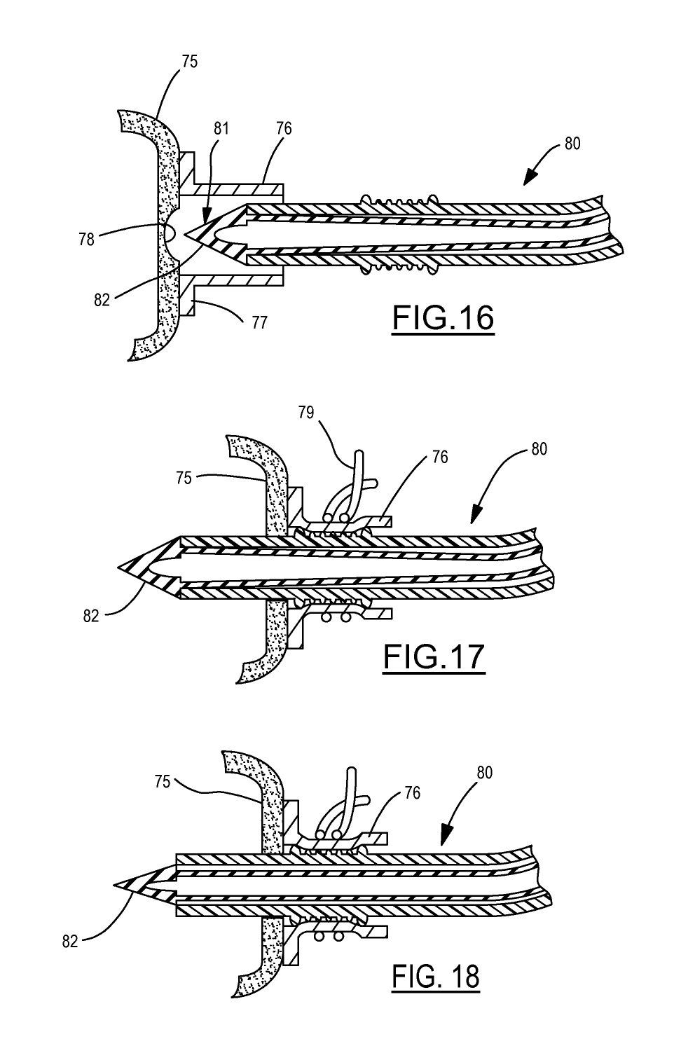

FIGS. 15-18 show surgical implantation of an inflow conduit using the expansion tool of the present invention in greater detail. Heart wall 75 has cuff 76 attached thereto. The plastic flange of cuff 76 is attached to heart wall 75 using sutures (not shown). After cuff 76 has been attached and the entry point of the inflow conduit is determined, a partial incision 78 is made to facilitate punching through wall 75. Inflow conduit 80 has expansion tool 81 mounted thereon and is inserted into cuff 76 so that tip 82 approaches incision 78. Tip 82 is forced through incision 78 to enter the heart through wall 75 as shown in FIG. 16. A ribbed portion of inflow conduit 80 enters cuff 76 so that a final attachment may be made by wrapping sutures 79 around cuff 76, resulting in a flexible connection that seals against any loss of blood. As shown in FIG. 17, tip 82 is moved into its contracted configuration which is adapted to provide a sliding seal along the cylindrical inside surface of inflow conduit 80. As shown on FIG. 18, expansion tool 81 is withdrawn and a sliding seal 83 advances through conduit 80. As blood enters via the distal end of inflow conduit 80, air exits from the proximal side of inflow conduit 80.

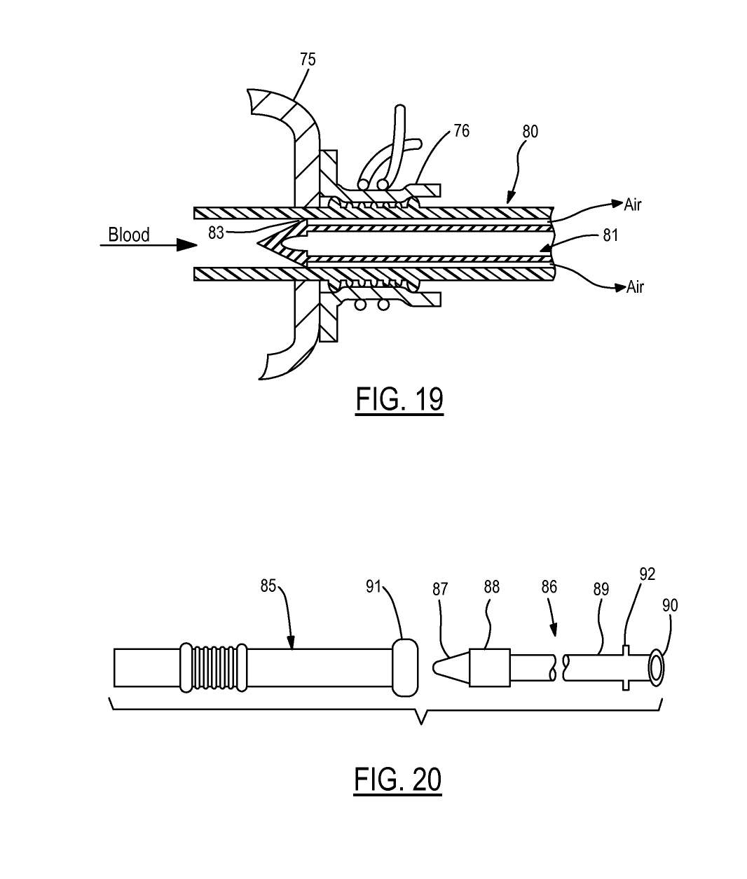

FIG. 19 illustrates an alternative embodiment of the invention using an inflow conduit 85 and a syringe-like plunger tool 86. Tip 87 is provided on tool 86 for punching through the heart tissue, and an intermediate sealing body 88 is provided at the base of tip 87. Sealing body 88 has a diameter greater than that of tip 87 and just slightly less than or equal to the inside diameter of inflow conduit 85. Sealing body 88 may be comprised of a resilient material for providing the sliding seal along the internal surface of inflow conduit 85. A flexible shaft member 89 couples tip 87 and sealing body 88 at the distal end to a gripping portion 90 at the proximal end of plunger tool 86. In this embodiment, plunger tool 86 functions like a syringe member. Tip 87 may have a substantially constant shape and a diameter less than or equal to that of sealing body 88. In order to maintain plunger tool 86 within inflow conduit 85 prior to use and during the application of punching force, a locking mechanism is provided that may include a knurl 91 on inflow conduit 85 that mates with a locking flange 92 on plunger tool 86.

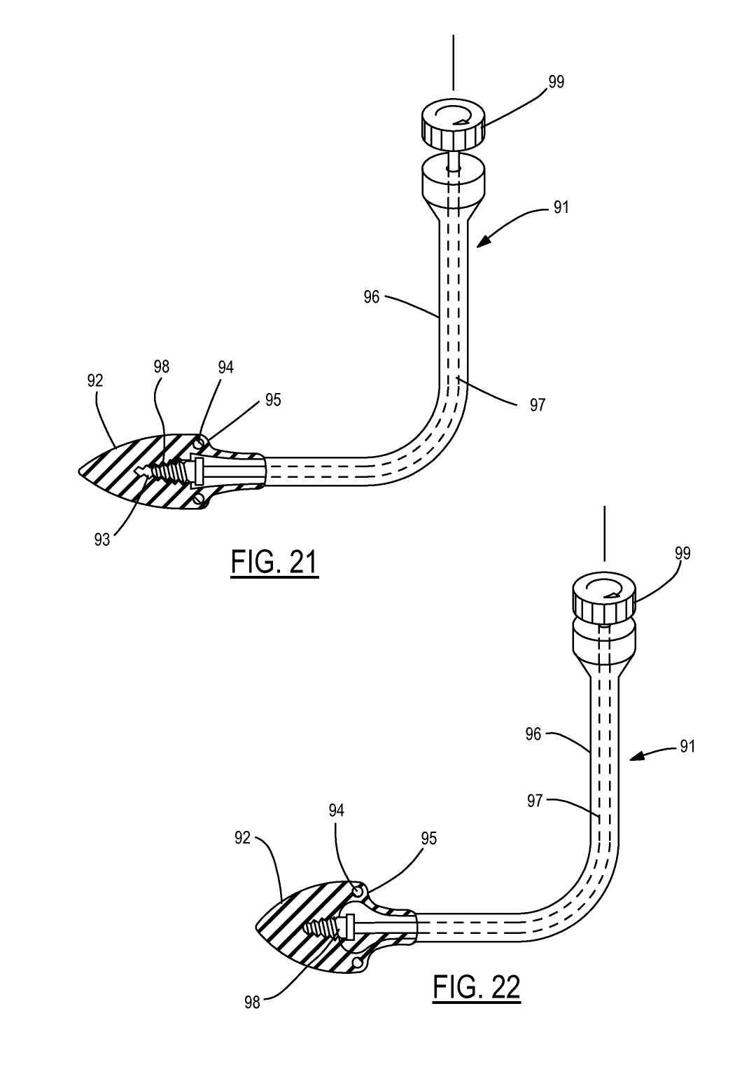

FIGS. 20 and 21 show another embodiment of the expansion tool using a tapered screw mechanism. Expansion tool 91 has a tip member 92 with internal female threads 93 and an embedded ring 94. Tip 92 may be comprised of soft silicone rubber and may have other embedded structures for controlling its shape or rigidity. Ring 94 is expandable to maintain a circle shape during expansion so that the overall shape at the base of tip 92 is maintained as desired. Specifically, an abutment surface 95 is provided behind ring 94 for interfacing with the distal end of the inflow conduit when expanded. A hollow, flexible body 96 is attached to the proximal end of tip 92 and receives an adjustment rod 97 having tapered screw 98 on one end and an adjustment knob 99 on the other end. In the contracted configuration in FIG. 20, tapered screw 98 is not fully inserted into female threads 93. By turning adjustment knob 99, the expanded configuration shown in FIG. 21 is obtained by advancing tapered screw 98 fully into female threads 93 in tip 92.

In all of the disclosed embodiments, the sliding seal is preferably comprised of a component (i.e., the tip or intermediate sealing body) made from a pliable material and preferably includes a lubricant coating.

Other expansion mechanisms for the blunt tip could also be employed such as a hydraulic mechanism.

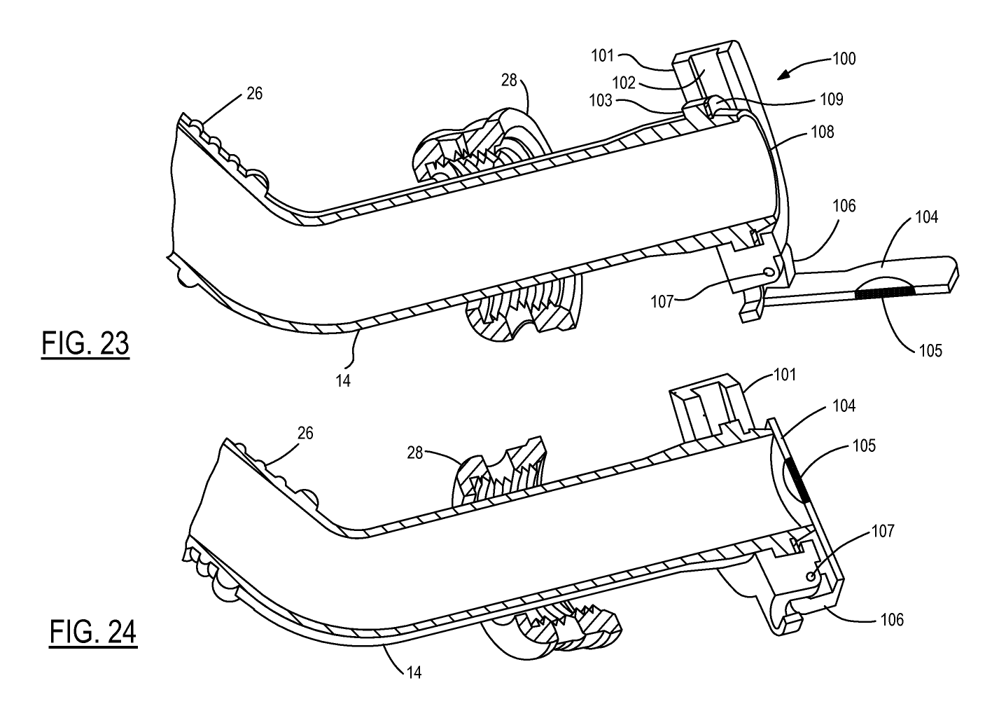

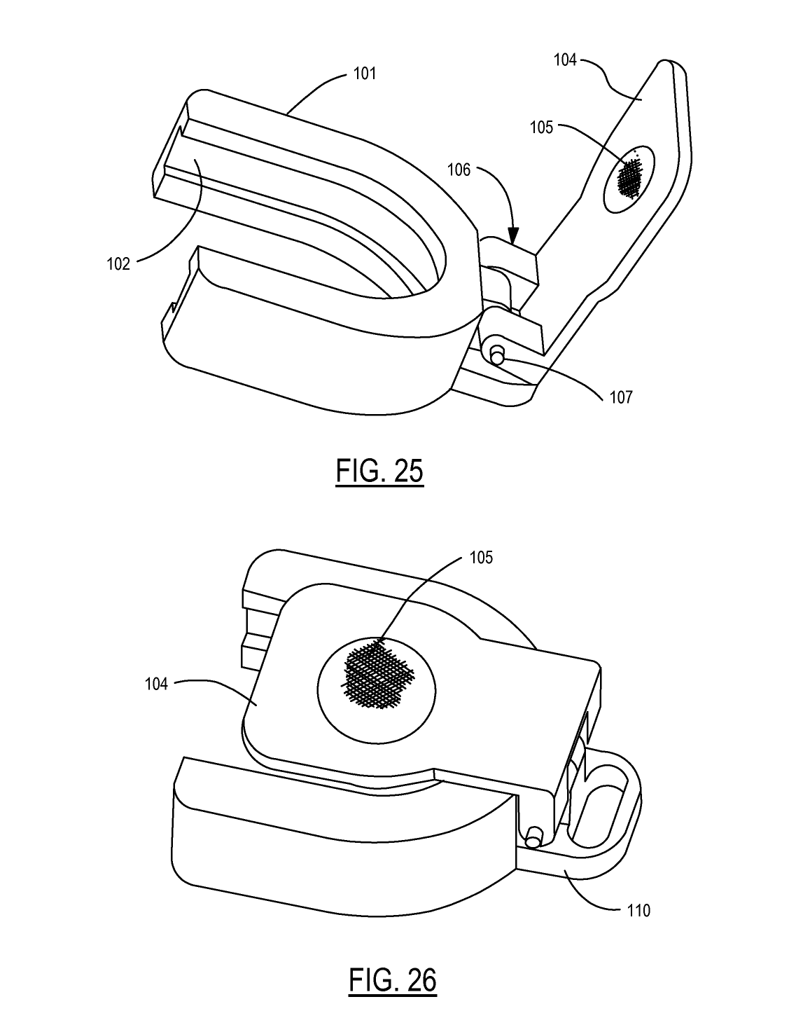

In a surgical procedure, there is a possibility that some time may elapse from the time that the inflow conduit is inserted to the heart and attached to the cuff until the time that the final connection of the inflow conduit to the pump is made. A further feature of the present invention includes a flap valve that is used to seal off the open end of the inflow conduit after the expansion tool is removed and until the inflow conduit is connected to the pump. A first embodiment is shown in an open position in FIGS. 22 and 24 and a closed position in FIGS. 23 and 25. A flap valve device 100 has a U-shaped base 101 that can be manually applied to and released from the end of conduit 14. Base 101 has a slot 102 for sliding onto a flange 103 formed at the end of inflow conduit 14. A planar flap member 104 extends from a block 106 that is pivotably linked to base 101 by a hinge pin 107. With flap member 104 in the open position as shown in FIG. 22, the expansion tool can reside within inflow conduit 14 during attachment to the heart wall and while it is subsequently withdrawn from inflow conduit 14. After removal of the expansion tool, flap member 104 is seated against an edge 108 of inflow conduit 14 to prevent loss of blood while waiting for the connection to the pump. Preferably, a spring (not shown) is incorporated into the pivotable mount of flap member 104 to automatically seat flap member 104 against edge 108 as soon as the expansion tool is out of the way.

Since inflow conduit 14 is filled with blood and is substantially free of air when the expansion tool is removed, very little air gets trapped when flap member 104 becomes seated. In order to vent any air that may accumulate, flap member 104 preferably has a vent window 105 comprised of a material that is impenetrable by blood but that allows passage of air. Various membranes or filter materials are well known within the field of blood treatment that will permit the migration of air but are impermeable to blood.

With flap member 104 in the closed position, continued beating of the heart does not result in loss of blood through the inflow conduit. When the surgeon is ready to attach the inflow conduit to the pump, U-shaped base 101 is pulled off of inflow conduit 14 by grasping a pull tab 110. Then threaded coupling 28 is attached to the pump. Flap valve device 100 may then be disposed of.

Other methods of attaching the flap valve device may be employed provided that the flap valve device is releasably mounted to the end of the inflow conduit for blocking blood flow from the inflow conduit after removal of the expansion tool. The flap valve device comprises a flap member with at least a portion made of an air-permeable material to allow escape of air from the inflow conduit when the flap member is seated against the end of the inflow conduit.

What is claimed is:

1. A method of attaching an inflow conduit and blood pump to a heart of a patient so that a distal end of the inflow conduit passes through a heart wall into a ventricle and a proximal end of the inflow conduit attaches to the blood pump, the method comprising the steps of:
installing a catheter with an inflation member into the inflow conduit so that the inflation member presses against and blocks the distal end of the inflow conduit;
installing a cuff onto the inflow conduit, wherein the cuff includes a tubular portion for receiving the inflow conduit and a flange portion for attaching to the heart;
partially incising the heart wall;
loosely coupling the flange portion using sutures extending between the flange portion and the heart wall near the partial incision;
piercing a hole through the heart wall at the partial incision into the ventricle using a blunt tool;
inserting the distal end of the inflow conduit through the hole;
closely coupling the flange portion to the heart wall using tightened sutures;
at least partially deflating the inflation member;
withdrawing the catheter from the inflow conduit to allow gradual entry of blood into the distal end and withdrawal of air from the proximal end; and
tightly securing the tubular portion of the cuff to the inflow conduit.

2. The method of claim 1 wherein the step of installing the cuff onto the inflow conduit occurs prior to loosely coupling the flange portion to the heart wall and includes tying a loose suture loop around the tubular portion.

3. The method of claim 1 wherein the catheter further includes a drain lumen, and wherein the method further comprises the step of draining air from within the ventricle after inserting the distal end of the inflow conduit through the hole.

4. A method of attaching an inflow conduit of a blood pump to a heart having a chamber, wherein the inflow conduit has a first end for inserting into the heart, an intermediate portion for attaching to the cuff, and a second end for conveying blood from the heart to the pump, the method comprising the steps of:
releasably mounting an expansion tool within the inflow conduit, wherein the expansion tool is longitudinally slidable within the inflow conduit, wherein the expansion tool has a blunt tip at a distal end and a manual grip at a proximal end;
inserting the first end into the chamber with the blunt tip projecting from the first end;
attaching the intermediate portion to the cuff;
pulling the expansion tool out from the inflow conduit so that a sliding seal substantially adjacent the blunt tip travels along the inflow conduit resulting in gradual entry of blood into the first end and withdrawal of air from the second end; and
attaching the second end to the blood pump.

5. Apparatus for connecting a pump to a chamber of a heart, wherein a cuff attaches to the heart, the cuff having a flange portion for attaching to a surface of the heart and a sleeve portion extending from the flange portion, the apparatus comprising:
an inflow conduit having a first end for inserting into the heart, an intermediate portion for attaching to the sleeve portion of the cuff, and a second end for conveying blood from the heart to the pump; and
an expansion tool releasably mounted with the inflow conduit to slide longitudinally within the inflow conduit, wherein the expansion tool has a blunt tip for punching through the heart into the chamber at a distal end and a manual grip at a proximal end for pulling the expansion tool out from the inflow conduit, and wherein the expansion tool has a sliding seal substantially adjacent the blunt tip so that after punching into the heart and joining the inflow conduit to the sleeve portion of the cuff, the expansion tool can be withdrawn from the inflow conduit resulting in gradual entry of blood into the first end and withdrawal of air from the second end.

6. The apparatus of claim 5 wherein the blunt tip has an expandable base with an expanded configuration and a contracted configuration, wherein the expandable base has a diameter in the expanded configuration greater than an inside diameter of the inflow conduit so that the expandable base abuts a forward edge of the first end of the inflow conduit when the blunt tip punches through the heart, and wherein the base provides the sliding seal when in the contracted configuration.

7. The apparatus of claim 6 wherein the expansion tool comprises an expansion mechanism within the blunt tip coupled to a manual adjuster at the proximal end.

8. The apparatus of claim 7 wherein the expansion mechanism is comprised of a tapered screw mechanism.

9. The apparatus of claim 7 wherein the expansion mechanism is comprised of a wedge mechanism.

10. The apparatus of claim 6 wherein the expandable base comprises a pliable material, wherein the inflow conduit has an anti-coagulant coating, wherein the expandable base provides the sliding seal along the anti-coagulant coating while in the contracted configuration, and wherein the expandable base has a diameter in the contracted configuration such that a gap is provided between the expandable base and the inflow conduit to prevent scrubbing of the anti-coagulant coating while maintaining a seal that substantially prevents air from entering the gap.

11. The apparatus of claim 10 wherein the expandable base further comprises a lubricant applied to the pliable material.

12. The apparatus of claim 5 wherein the inflow conduit has an intermediate bend, and wherein the expansion tool has a flexible intermediate section between the distal end and the proximal end.

13. The apparatus of claim 12 wherein the intermediate section includes a coiled tubular member.

14. The apparatus of claim 5 wherein the blunt tip has a generally conical shape.

15. The apparatus of claim 5 wherein the blunt tip has a generally duck-bill shape.

16. The apparatus of claim 5 wherein the expansion tool is comprised of a syringe member, wherein the blunt tip has a substantially constant shape and a diameter less than or equal to an inside diameter of the inflow conduit, and wherein the syringe member includes an intermediate sealing body to provide the sliding seal.

17. The apparatus of claim 16 wherein the intermediate sealing body is comprised of a pliable material.

18. The apparatus of claim 16 wherein the expansion tool includes a releasable locking mechanism to fix the expansion tool with respect to the inflow conduit while the blunt tip punches through the heart.

19. The apparatus of claim 5 further comprising a flap valve device releasably mounted to the second end of the inflow conduit for blocking blood flow from the second end of the inflow conduit after removal of the expansion tool.

20. The apparatus of claim 19 wherein the flap valve device comprises a flap member comprised of an air-permeable material allowing escape of air from the inflow conduit when the flap member is seated against the second end.

21. A surgical tool for use in attaching an inflow conduit of a blood pump to a heart, wherein a cuff is attached to the heart, and wherein the inflow conduit has a first end for inserting into the heart, an intermediate portion for attaching to the cuff, and a second end for conveying blood from the heart to the pump, wherein the surgical tool comprises:
an expansion tool for releasably mounting within the inflow conduit to slide longitudinally within the inflow conduit, wherein the expansion tool has a blunt tip for punching through the heart at a distal end and a manual grip at a proximal end for pulling the expansion tool out from the inflow conduit, and wherein the expansion tool has a sliding seal substantially adjacent the blunt tip so that after punching into the heart and joining the inflow conduit to the cuff, the expansion tool can be withdrawn from the inflow conduit resulting in gradual entry of blood into the first end and withdrawal of air from the second end.

22. The tool of claim 21 wherein the blunt tip has an expandable base with an expanded configuration and a contracted configuration, wherein the expandable base has a diameter in the expanded configuration greater than an inside diameter of the inflow conduit so that the expandable base can abut a forward edge of the first end of the inflow conduit when the blunt tip punches through the heart, and wherein the base provides the sliding seal when in the contracted configuration.

23. The tool of claim 22 wherein the expansion tool comprises an expansion mechanism within the blunt tip coupled to a manual adjuster at the proximal end.

24. The tool of claim 23 wherein the expansion mechanism is comprised of a tapered screw mechanism.

25. The tool of claim 23 wherein the expansion mechanism is comprised of a wedge mechanism.

26. The tool of claim 22 wherein the expandable base comprises a pliable material.

27. The tool of claim 21 wherein the expansion tool has a flexible intermediate section between the distal end and the proximal end.

28. The tool of claim 27 wherein the intermediate section includes a coiled tubular member.

29. The tool of claim 21 wherein the blunt tip has a generally conical shape.

30. The tool of claim 21 wherein the blunt tip has a generally duck-bill shape.

31. The tool of claim 21 wherein the expansion tool is comprised of a syringe member, wherein the blunt tip has a substantially constant shape and a diameter less than or equal to an inside diameter of the inflow conduit, and wherein the syringe member includes an intermediate sealing body to provide the sliding seal.

32. The tool of claim 31 wherein the intermediate sealing body is comprised of a pliable material with a lubricant coating.

33. The tool of claim 21 wherein the inflow conduit has an anti-coagulant coating, wherein the expandable base provides the sliding seal along the anti-coagulant coating while in the contracted configuration, and wherein the expandable base has a controlled diameter in the contracted configuration such that a gap is provided between the expandable base and the inflow conduit to prevent scrubbing of the anti-coagulant coating while maintaining a seal that substantially prevents air from entering the gap
